(19) United States
(12) Patent Application Publication
(10) Pub. No.: US 2006/0128637 A1
(43) Pub. Date: Jun. 15, 2006

(54) Phenolic acid complexes of hyoscyamine and process for preparing the same

(76) Inventors: Jeffrey S. Kiel, Gainesville, GA (US); H. Greg Thomas, Carrollton, GA (US); Emily Childrey Ware, Athens, GA (US); Brady Neal Ware, Athens, GA (US)

Correspondence Address: Marla J. Church, Esq.;Kiel Laboratories, 1233 Palmour Drive, Gainesville, GA, 30501, US

(21) Appl. No.: 11/302,577

(22) Filed: Dec. 14, 2005

Related U.S. Application Data
(60) Provisional application No. 60/636,191, filed on Dec. 15, 2004.

(51) Int. Cl. C07D 451/02; A61K 31/7024; A61K 31/46

(52) U.S. Cl. 514/23; 514/304; 546/124

(57) ABSTRACT

The present invention provides a novel tannate complex of hyoscyamine for human and veterinary pharmaceutical use. Tannate complexes of active pharmaceutical ingredients are used in sustained release applications and to improve certain organoleptic properties such as taste. A process for preparing the tannate complex of hyoscyamine is provided.

This application claims the benefit of U.S. Provisional Patent Application Ser. No. 60/636,191 filed on Dec. 15, 2004.

### BACKGROUND OF THE INVENTION

1. Field of the Invention

The present invention relates generally to the field of phenolic acid chemistry and, more particularly, to the field of tannate chemistry and the compound hyoscyamine tannate.

2. Description of the Prior Art

Hyoscyamine is a natural anti-muscarinic alkaloid isolated from plants of the Solanaceae family such as Hyoscyamus niger, Atropa belladonna, and Datura stramonium. Hyoscyamine is used to treat rhinitis, bladder spasms, diverticulitis, colic, irritable bowel syndrome, cystitis, pancreatitis, and is indicated as adjunctive therapy in the treatment of peptic ulcers. Hyoscyamine is a crystalline solid and is freely soluble in water. Chemically, hyoscyamine is [3(S)-endo]-α-(Hydroxymethyl)-benzeneacetic acid 8-methyl-8-azabicyclo[3.2.1]oct-3-yl ester with the empirical formula C₁₇H₂₃NO₃ and a MW of 289.36. Typically hyoscyamine is administered in multiple doses for optimal pharmacological action.

Tannic acid is one example of a phenolic acid compound. Naturally occurring tannic acid comprises a mixture of compounds. They are considered to be secondary metabolites, with a molecular weight of 500-5000 Da, that have no specific metabolic function. As with many natural polymers, a rigorous chemical definition of tannins is difficult. Tannic acid drug complexes are commonly referred to as tannates or tannate complexes.

Tannate complexes have been found to have better organoleptic properties such as taste in comparison to other complexes or free base forms. In comparison to typical complex forms, the tannate complex of the active pharmaceutical ingredient (API) is significantly larger molecule that is typically less soluble, which affords absorption of the API over prolonged intervals of time, reducing the frequency of administration and thereby improving patient compliance. There is no suggestion of the preparation of hyoscyamine tannate in the prior art.

### SUMMARY OF THE INVENTION

In accordance with the object of the present invention a chemical composition comprising hyoscyamine tannate is provided. A further object of the present invention is to provide a chemical complex comprising hyoscyamine tannate and magnesium aluminum silicate (MAS). Further, a process for preparing the novel and useful chemical compound hyoscyamine tannate is provided. A process for preparing hyoscyamine tannate and MAS complex is further described as comprising the mixing of hyoscyamine (in salt or free base form), MAS, and tannic acid together in dry powder form or in the presence of one or more solvents. The method may further include the step of selecting the one or more solvents from a group consisting of purified water, ethanol, glycerin, propylene glycol, diethylether, methylene chloride, acetone, isopropyl alcohol and mixtures thereof.

The process may also include the steps of isolating and purifying the tannate complex. This may be accomplished by filtration, drying, centrifugation and lyophilization.

Tannic acid is one example of a phenolic acid compound. Naturally occurring tannic acid comprises a mixture of compounds. They are considered to be secondary metabolites, with a molecular weight of 500-5000 Da, that have no specific metabolic function. As with many natural polymers, a rigorous chemical definition of tannins is difficult.

Hydrolyzable tannins are molecules with a polyol (generally D-glucose) as a central core, with the hydroxyl groups of the carbohydrate partially or totally esterified with phenolic groups. They derive their name from their propensity to be hydrolyzed by mild acids or mild bases to yield carbohydrates and phenolic acids. Synthetic tannic acid may comprise a purified form of any of the components of naturally occurring tannic acid.

The present invention may utilize tannic acid of either a natural or synthetic source. The term “tannic acid” herein refers to either natural or synthetic tannic acid as described above.

Magnesium aluminum silicate (MAS) may be from a natural or synthetic source that is variable in composition among different sources. It is a substance commonly used in pharmaceutical preparations as a dispersing and suspending agent. MAS as referenced in the present application means MAS from any of the available sources.

### DETAILED DESCRIPTION OF THE INVENTION

The present invention relates to a novel compound and composition comprising hyoscyamine tannate as well as a novel method of preparing the tannate complex of the active pharmaceutical ingredient (API) hyoscyamine. Hyoscyamine is an anticholinergic agent used to treat rhinitis, bladder spasms, diverticulitis, colic, irritable bowel syndrome, cystitis, pancreatitis, and is indicated as adjunctive therapy in the treatment of peptic ulcers. Hyoscyamine is a crystalline solid and is freely soluble in water. Chemically, hyoscyamine is [3(S)-endo]-α-(Hydroxymethyl)-benzeneacetic acid 8-methyl-8-azabicyclo[3.2.1]oct-3-yl ester with the empirical formula C₁₇H₂₃NO₃ and a MW of 289.36. Typically hyoscyamine is administered in multiple doses for optimal pharmacological action.

The literature describes many ways of preparing hyoscyamine from a variety of starting materials, but there is no suggestion of the preparation of hyoscyamine tannate.

As discussed previously, tannic acid is one example of a phenolic acid compound. Naturally occurring tannic acid comprises a mixture of compounds. Naturally occurring tannic acid comprises a mixture of compounds. They are considered to be secondary metabolites, with a molecular weight of 500-5000 Da, that have no specific metabolic function. They are complex phenol-rich polymers found in many foods. As with many natural polymers, a rigorous chemical definition of tannins is difficult. In general two classes are distinguished—the hydrolyzable and the condensed tannins. Hydrolyzable tannins or tannic acids are referenced in the various pharmacopeias and are typically composed of a phenolic acid such as gallic acid or its condensation product ellagic acid esterified to the hydroxyl groups of a polyol such as glucose or quinic acid. They derive their name from their propensity to be hydrolyzed by mild acids or mild bases to yield carbohydrates and phenolic acids. Synthetic tannic acid may comprise a purified form of any of the components of naturally occurring tannic acid.

The present invention may utilize tannic acid of either a natural or synthetic source. The term “tannic acid” herein refers to either natural or synthetic tannic acid as described above. Whenever an active pharmaceutical ingredient (API) is mentioned in this specification, it should be understood that the statement can refer to either the free base or salt form.

Magnesium aluminum silicate (MAS) may also be from a natural or synthetic source that is variable in composition among different sources. It is a substance commonly used in pharmaceutical preparations as a dispersing and suspending agent. MAS as referenced in the present application means MAS from any of the available sources.

Tannate complexes have been found to have better organoleptic properties such as taste in comparison to other complexes or free base forms. In comparison to typical complex forms, the tannate complex of the active pharmaceutical ingredient (API) is a significantly larger molecule that is typically less soluble, which affords absorption of the API over prolonged intervals of time, reducing the frequency of administration and thereby improving patient compliance.

Specifically describing the present process of preparing hyoscyamine tannate and MAS complex, the process comprises the mixing of hyoscyamine (in salt or free base form), MAS, and tannic acid in the presence of a solvent. More specifically, the mixing step further includes adding the hyoscyamine (in salt or free base form) to a solvent, adding MAS, then adding tannic acid to the hyoscyamine (in salt or free base form), MAS, and the solvent. Alternatively, the mixing step may include adding the tannic acid to a solvent and then adding MAS, then adding hyoscyamine (in salt or free base form) to the tannic acid, solvent and MAS. In yet another alternative, the mixing step may be described as including the mixing of hyoscyamine (in salt or free base form), tannic acid, and MAS powders together and then the adding of a solvent. Still further, the mixing may include adding hyoscyamine (in salt or free base form) to a solvent to create a first reaction mixture, adding tannic acid and MAS to a solvent to create a second reaction mixture and mixing together the two reaction mixtures.

Still further the process may be described as including the additional requirement of maintaining a temperature of between about 15 and about 150 degrees C. during the conversion. The process may also be further defined as including the requirement of maintaining a pH of between about 2 and about 11 during the conversion. Additionally, the method may be further described as including the requirement that the weight ratio of tannic acid to hyoscyamine be between about 0.1:1 to about 20:1. Additionally, the method may be further described as including the requirement that the weight ratio of MAS to hyoscyamine be between about 0.1:1 to about 20:1.

The process may also include the steps of isolating and purifying the tannate complex. This may be accomplished by filtration, drying, centrifugation and lyophilization.

The invention will further be described by means of the following Examples, which illustrate the preparation of hyoscyamine tannate.

### EXAMPLE 1

### Formation of the Insoluble Tannate Complex—Dispersion Method—I

| Ingredient | Amount (g) | |
|---|---|---|
| Hyoscyamine | 40.000 | |
| Tannic acid | 48.000 | |
| Purified water | 450 | ml |

The conversion process used in this Example to prepare hyoscyamine tannate is done as follows. About 150 ml of purified water is placed in a suitable vessel and hyoscyamine is added to the water and stirred to form a solution. Three hundred milliliters of purified water is placed in a separate bowl. While mixing the water, tannic acid is added and mixing is continued to form a dispersion. While mixing the tannic acid, the solution of hyoscyamine is added slowly. Once all of the hyoscyamine solution is added, mixing is continued for 10-15 minutes. The preparation yields hyoscyamine tannate as a precipitated complex. The ratio of tannic acid to hyoscyamine used in this Example is 1.2:1. The complex is then recovered by filtration and drying.

The completeness of the reaction and the formation of the tannate complex are followed by taking samples at different stages of the conversion process. Due to the large size of the tannate complex, it usually precipitates from solution upon formation. The absence or loss of hyoscyamine from solution is correlated with the formation of the tannate complex.

### EXAMPLE 2

### Formation of the Insoluble Tannate Complex—Dispersion Method—II

| Ingredient | Amount (g) | |
|---|---|---|
| Hyoscyamine | 40.000 | |
| Tannic acid | 48.000 | |
| MAS | 48.000 | |
| Purified water | 450 | ml |

The conversion process used in this Example to prepare hyoscyamine tannate is done as follows. About 150 ml of purified water is placed in a suitable vessel and hyoscyamine is added to the water and stirred to form a solution. Three hundred milliliters of purified water is placed in a separate bowl. While mixing the water, tannic acid and MAS are added and mixing is continued to form a dispersion. While mixing the tannic acid, MAS dispersion, the solution of hyoscyamine is added slowly. Once all of the hyoscyamine solution is added, mixing is continued for 10-15 minutes. The preparation yields hyoscyamine tannate as a precipitated complex. The ratio of tannic acid to hyoscyamine used in this Example is 1.2:1. The complex is then recovered by filtration and drying.

The completeness of the reaction and the formation of the tannate complex are followed by taking samples at different stages of the conversion process. Due to the large size of the tannate complex, it usually precipitates from solution upon formation. The absence or loss of hyoscyamine from solution is correlated with the formation of the tannate complex.

### EXAMPLE 3

### Formation of the Insoluble Tannate Complex—Dispersion Method—III

| Ingredient | Amount (g) | |
|---|---|---|
| Hyoscyamine | 40.000 | |
| Tannic acid | 32.000 | |
| MAS | 32.000 | |
| Purified water | 250 | ml |
| Ethanol | 50 | ml |

The conversion process used in this Example to prepare the tannate complex of hyoscyamine can be done as follows: 250 ml of purified water and 50 ml of ethanol are placed in a beaker. While mixing the water/ethanol mixture, tannic acid and MAS are added and mixing is continued to form a dispersion. While mixing the tannic acid, MAS dispersion, hyoscyamine powder is added slowly. The hyoscyamine reacts with the tannic acid to form the tannate complex. Since the tannate complex has low solubility, it is precipitated from solution and can be isolated by centrifugation. The ratio of tannic acid to hyoscyamine used is 0.8:1.

The completeness of the reaction and the formation of the tannate complex can be followed as described in Example 1.

### EXAMPLE 4

### Formation of the Insoluble Tannate Complex—Dry Powder Method—I

| Ingredient | Amount (g) | |
|---|---|---|
| Hyoscyamine | 88.24 | |
| Tannic acid | 88.24 | |
| MAS | 88.24 | |
| Purified water | 150 | ml |

The conversion process used in this Example to prepare the tannate complex of the active is performed using the following procedure. About 150 ml of purified water is placed in a suitable vessel and hyoscyamine is added to the water and stirred to dissolve. Tannic acid and MAS powders are placed in a suitable planetary mixer. While mixing the tannic acid and MAS powders, the solution of hyoscyamine is added to generate the hyoscyamine tannate. The ratio of tannic acid to hyoscyamine used is 1:1. Experiments similar to those used to assay formation of the tannate complex in Examples 1 and 2 can be applied to this Example as well.

### EXAMPLE 5

### Formation of the Insoluble Tannate Complex—Dry Powder Method—II

| Ingredient | Amount (g) | |
|---|---|---|
| Hyoscyamine | 88.24 | |
| Tannic acid | 176.48 | |
| MAS | 176.48 | |
| Purified water | 100 | ml |

The conversion process used in this Example to prepare the tannate complex of the active is performed using the following procedure. Hyoscyamine, tannic acid, and MAS powders are placed in a suitable planetary mixer or blender and mixed for a period of 10 minutes to obtain a powder blend. 100 mL of purified water is added onto the powders while mixing, to generate the tannate complex of hyoscyamine. This synthetic process yields hyoscyamine tannate complex as a uniformly distributed, lump free powder mass.

The ratio of tannic acid to hyoscyamine used is 1:2. Experiments similar to those used to assay formation of the tannate complex in Examples 1 and 2 can be applied to this Example as well.

### EXAMPLE 6

### Formation of the Insoluble Tannate Complex—Dry Powder Method—III

| Ingredient | Amount (g) |
|---|---|
| Hyoscyamine | 88.24 |
| Tannic acid | 176.48 |

The conversion process used in this Example to prepare the tannate complex of the active can be performed using the following procedure. Hyoscyamine and tannic acid powders are placed in a suitable planetary mixer or blender and mixed at a temperature of 95 degrees C. Mixing is continued for 30 minutes at the high temperature to prepare hyoscyamine tannate.

The ratio of tannic acid to hyoscyamine used is 1:2. Experiments similar to those used to assay formation of the tannate complex in Examples 1 and 2 can be applied to this Example as well.

### EXAMPLE 7

### Formation of the Insoluble Tannate Complex—Dry Powder Method—IV

| Ingredient | Amount (g) |
|---|---|
| Hyoscyamine | 88.24 |
| Tannic acid | 176.48 |
| MAS | 176.48 |

The conversion process used in this Example to prepare the tannate complex of the active can be performed using the following procedure. Hyoscyamine and tannic acid powders are placed in a suitable planetary mixer or blender and mixed at a temperature of 95 degrees C. Mixing is continued for 30 minutes at the high temperature to prepare hyoscyamine tannate.

The ratio of tannic acid to hyoscyamine used is 1:2. Experiments similar to those used to assay formation of the tannate complex in Examples 1 and 2 can be applied to this Example as well.

It should be understood that the above Examples are illustrative of several different embodiments of the invention herein disclosed. Given the present disclosure, it is anticipated that numerous variations will be devised by those skilled in the art without departing from the spirit and scope of the present invention. Latitude of modification, substitution and change is intended and in some instances, some features of the invention will be employed without a corresponding use of other features. Accordingly, it is intended that the spirit and scope of the invention disclosed herein should be limited only by the following claims.

We claim:

1. Hyoscyamine tannate

2. The hyoscyamine tannate of claim 1, wherein the tannate is derived from a tannic acid of either natural or synthetic origin.

3. Hyoscyamine tannate wherein said hyoscyamine tannate is formed by mixing hyoscyamine and tannic acid together using a weight ratio of said tannic acid to said hyoscyamine of between 0.1:1 to about 20:1.

4. The hyoscyamine tannate of claim 3, wherein the tannic acid is derived from either natural or synthetic origin.

5. Hyoscyamine, tannic acid and magnesium aluminum silicate (MAS) complex.

6. The complex of claim 5, wherein the tannic acid is of either natural or synthetic origin.

7. A process for preparing a hyoscyamine, tannic acid and MAS complex comprising: mixing hyoscyamine, MAS, and tannic acid in the presence of one or more solvents.

8. The process of claim 7, wherein the one or more solvents are selected from a group consisting of purified water, ethanol, glycerine, propylene glycol, diethylether, methylene chloride, acetone, isopropyl alcohol and mixtures thereof.

9. The process of claim 7, wherein said mixing step includes adding the hyoscyamine to the one or more solvents, adding MAS, then adding tannic acid to the hyoscyamine, MAS, and the one or more solvents.

10. The process of claim 7, wherein said mixing step includes adding the tannic acid to the one or more solvents, adding MAS, adding hyoscyamine to the tannic acid, MAS, and the one or more solvents.

11. The process of claim 7, wherein said mixing step includes the mixing of hyoscyamine, tannic acid and MAS in powder form together and then adding the one or more solvents.

12. The process of claim 7, wherein said mixing step includes adding said hyoscyamine to the one or more solvents to create a first reaction mixture, adding tannic acid and MAS to one or more solvents to create a second reaction mixture and mixing together said first and second reaction mixtures.

13. The process of claim 7, including mixing said hyoscyamine, MAS, and tannic acid together while maintaining a temperature of between about 15 to about 150 degrees C.

14. The process of claim 7, wherein said mixing is performed while maintaining a pH of between about 2 and about 11.

15. The process of claim 7, including using a weight ratio of said tannic acid to said hyoscyamine of between about 0.1:1 to about 20:1.

16. The process of claim 7, including using a weight ratio of said MAS to said hyoscyamine of between about 0.1:1 to about 20:1.

17. The process of claim 7, further including isolating and purifying hyoscyamine tannate.

18. The process of claim 17, including performing said isolating and purifying step by a procedure selected from a group consisting of filtering, drying, centrifuging and lyophilizing.
